(19) United States
(12) Patent Application Publication
(10) Pub. No.: US 2010/0331690 A1
(43) Pub. Date: Dec. 30, 2010

(54) Systems and Techniques for Providing Elasticity Graphs

(75) Inventors: Geng Li, Heng Fa Chuen (CN); Ying Zheng, Wuhan (CN); Edward S. Yang, Shatin (CN)

Correspondence Address: FULBRIGHT & JAWORSKI L.L.P, 2200 ROSS AVENUE, SUITE 2800, DALLAS, TX, 75201-2784, US

(73) Assignee: Hong Kong Applied Science and Technology Research Institute Co., Ltd., Shatin (CN)

(21) Appl. No.: 12/495,424

(22) Filed: Jun. 30, 2009

(51) Int. Cl. A61B 8/00

(52) U.S. Cl. 600/443

(57) ABSTRACT

An elastogram device comprises a driver controller including a wave generator, and an amplifier assembly receiving waves from the wave generator for driving an actuator, the amplifier assembly compatible with at least one pneumatic actuator, at least one hydraulic actuator, at least one piezoelectric actuator, and at least one electromechanical actuator, an elastogram processor receiving a wave image from an imaging source and generating an elastogram from the wave image, and a user input and output assembly including a display rendering the elastogram, a user interface providing a plurality of options for selection, the options including selectable parameters to control the wave generator and to generate the elastogram.

### CROSS-REFERENCE TO RELATED APPLICATIONS

The present application is related to U.S. patent application Ser. No. 12/194,949, filed Aug. 20, 2008, entitled, “PIEZOELECTRIC MAGNETIC RESONANCE ELASTOGRAPH (MRE) DRIVER SYSTEM,” the disclosure of which is hereby incorporated by reference herein.

### TECHNICAL FIELD

The present description relates, generally, to medical imaging and, more specifically, to methods and systems for generating elastograms.

### BACKGROUND OF THE INVENTION

Magnetic Resonance Elastography (MRE) is an MRI-based method for imaging the mechanical properties of tissue. The technique is used to depict the spatial distribution of tension in skeletal muscle, brain tissue, breast tissue, liver tissue, prostate tissue, etc. In this technique, a driver, e.g., pneumatic or electromechanical driver, is used to generate shear waves in a region of interest, such as brain, breast, liver, prostate, etc. of a human subject, while the human subject is located in a magnetic resonance imaging (MRI) system. In some instances, shear waves are generated by applying mechanical motion to the surface of the region of interest of the human subject. A mechanical actuator is coupled to the human subject, and provides cyclic motion that is synchronized to the MRI imaging sequence. Another way to generate shear waves in the tissue is to use a piezoelectric bending element. In other instances, a needle is inserted into the tissue of the animal or human subject, and the waves are generated by vibrating the needle. For more information about piezoelectric drivers, see Chan, Q. C. C. et al., “Localized Application of Shear Waves to Tissues for MR Elastography via a Needle Device,” Proceedings of the 13^{th} ISMRM, Florida, USA May 7-13, 2005; Chan, C. C., et al., “Shear Waves Induced by Moving Needle in MR Elastography, Proceedings of the 26^{th} Annual International Conference of the IEEE EMBS, San Francisco, Calif. USA, Sep. 1-5, 2004, pg. 1-3; Chan, Q. C. C., et al. “Needle Shear Wave Driver for Magnetic Resonance Elastography,” Magnetic Resonance in Medicine 55:1175-1179 (2006); Chen, Jun, et al., “Imaging Mechanical Shear Waves Induced by Piezoelectric Ceramics in Magnetic Resonance Elastography,” http://scholar.ilib.cn/Abstract.aspx?A=kxtb-e200606016, (downloaded Jun. 19, 2008); the disclosures of which are hereby incorporated herein by reference.

A technique referred to as Ultrasound Elastography (USE) is similar to MRE (described above), but instead of using an MRI imaging device, the technique uses an ultrasound imaging device while subjecting the patient's tissue to shear waves. The data is collected and an elastogram is generated, which indicates elasticity of tissue. While ultrasonic images are typically not as good as MRI images, ultrasound is often good enough when deciding whether to go the next step (e.g., biopsy). Elastograms can be used as a diagnostic tool as well as a screening tool. The typical goal of using an elastogram is the early detection of cirrhosis of the liver, breast cancer, and Alzheimer's disease in the early stage, usually through the detection of characteristics of stiffness or elasticity of tissue. X-ray mammograms are used to aid in the early detection and diagnosis of breast diseases in women, and elastograms are a potential replacement therefor. For instance, X-ray mammograms usually have a higher rate of false positives than do ultrasound elastograms. Fiber scans, which are used to detect cirrhosis of the liver, are also possible candidates for replacement by elastograms. However, elastogram technology is in its early stages, and elastogram devices are typically limited to laboratory systems built by researchers out of other separate pieces of laboratory equipment. There is currently no stand-alone elastogram device available on the market.

### BRIEF SUMMARY OF THE INVENTION

Various embodiments of the invention include elastogram devices that combine a wave excitation function, auto post-processing functions, and display and monitor systems into one commercial product. Some embodiments are portable and able to interface with a variety of imaging systems, MRI and ultrasound systems, and to actuate any of a variety of drivers, such as pneumatic drivers, hydraulic drivers, piezoelectric drivers, and electromechanical drivers.

According to one embodiment, a system is integrated in a single, portable device, which can be moved from room-to-room and building-to-building, even between and among healthcare institutions. The system has a driver controller including a wave generator capable of generating any of a variety of waveforms with a range of burst counts and frequencies. The wave generator also includes a user input/output (I/O) assembly for controlling the wave generator, the user I/O assembly including, e.g., a keypad or other device for entering information and a screen for showing system settings and real-time visualizations of the generated waves. The wave generator also includes an input for receiving synchronization information for the wave generator from an imaging source, such as an MRI device or an ultrasound device. Synchronization allows the elastogram device to coordinate the shear waves produced by the driver with the imaging acquisition of the imaging device. The wave generator also includes an amplifier assembly that receives waves from the wave generator for driving a plurality of types of actuators.

Further in this example embodiment, the system includes an imaging engine that has an elastogram processor with an MRI system-compatible and ultrasound system-compatible input receiving wave images from the imaging source. The elastogram processor generates an elastogram from the wave image by transforming the wave image to derive and visualize elasticity information. The system also includes a user input and output assembly that has a display rendering the elastogram and a user interface providing a plurality of options for selection, the options including selectable parameters (e.g., system settings, properties of the tissue under test, and the like) to control the wave generator and to generate the elastogram.

According to another embodiment, there exists a technique for use of an elastogram system. The elastogram system is communicatively coupled to the imaging source, both to receive wave images and to receive synchronization information. The type of imaging source is discerned so that the elastogram system stores data describing the imaging source (MRI, ultrasound). The wave generator actuates the driver to palpate the tissue as the imaging source generates imaging data of the tissue. The output of the imaging source as it images the tissue subjected to shear waves can be referred to as “wave image data” or simply “wave image.” The wave image data is received by the elastogram device and is provided to a post processing utility along with one or more of the parameters referred to above. The post processing utility uses the wave image data and the received parameters to generate the elastogram. The system then renders the elastogram on a display, such as a computer monitor or other display device.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 is an illustration of an exemplary system, adapted according to one embodiment of the invention;

FIG. 2 is an illustration of an exemplary system, adapted according to one embodiment of the invention;

FIG. 3 is an illustration of an exemplary keyboard controller, adapted according to one embodiment of the invention;

FIG. 4 is a block diagram of components of an exemplary keyboard controller according to one embodiment of the invention; and

FIG. 5 is an illustration of an exemplary method, adapted according to one embodiment of the invention, for generating an elastogram.

### DETAILED DESCRIPTION OF THE INVENTION

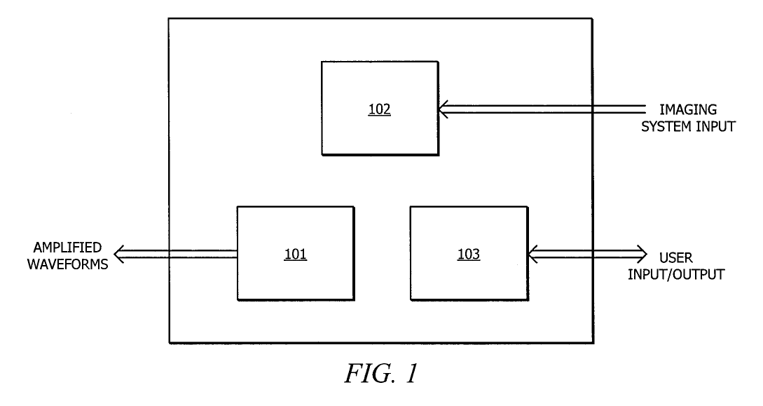

FIG. 1 is an illustration of exemplary system 100, adapted according to one embodiment of the invention. System 100 is an integrated, portable elastogram device including driver controller 101, which outputs amplified waveforms to one or more actuators that apply shear waves to tissues. Such actuators are often referred to as “drivers” and are not shown in FIG. 1 for ease of illustration. System 100 also includes elastogram processor 102, which receives imaging system output from an MRI system and/or an Ultrasound (US) system and generates an elastogram therefrom. Elastogram processor 102 can receive and process any of a variety of types of MRI and US data, including B-scan US data and Doppler US data.

Specifically, for both MR Elastography (MRE) and Ultrasound Elastography (USE), system 100 actuates the drivers to provide controllable shear waves in the object under investigation. Various parameters, such as waveform shape and frequency can be set automatically or manually. The waveforms can be synchronized with MRI or US scanners, and a real-time oscilloscope view of the applied waveform can be displayed to a user to verify proper operation. Additionally, system 100 includes user I/O assembly 103, which displays elastogram images to a user and also interacts with the user to output other information and receive user input. The various functional units of system 100 are described in more detail below.

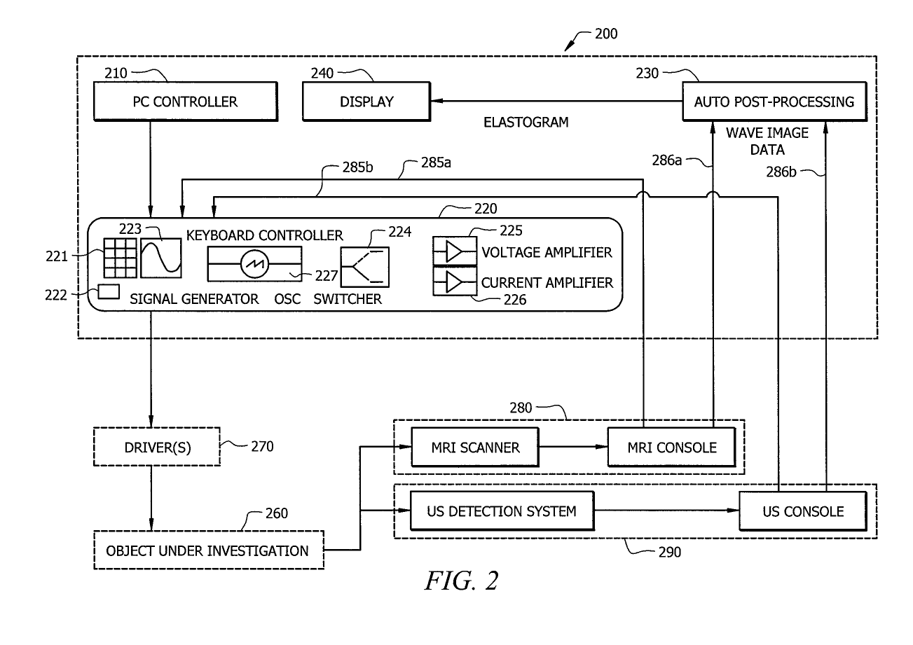

FIG. 2 is an illustration of exemplary system 200, adapted according to one embodiment of the invention. System 200 is an integrated elastogram device that is in communication with one or both of MRI system 280 and/or US system 290 and one or more drivers 270.

System 200 includes Personal Computer (PC) controller 210, which allows a user to interact with system 200 and to control the operation of system 200. In this example, PC controller 210 includes an intuitive and user-friendly interface that allows, among other things, for selection between operating modes. In an example mode referred to herein as a “clinic” mode, a user can input parameters known by the user, such as, e.g., tissue type (e.g., brain, liver, breast) and driver type (e.g., piezoelectric, pneumatic, hydraulic, electromechanical, or make/model), and PC controller 210 displays for selection and/or implements a number of suggested settings for system 200 and/or automatically implements those suggested settings. Examples of settings of system 200 that can be determined by user-input parameters include, e.g., excitation wave frequency, excitation wave burst count, pulse sequence, and output current and/or voltage for driver 270. Another mode, referred to herein as “research” mode, includes manual entry of system settings. Research mode may be used, for example, when a user desires system settings that are not necessarily accessible in clinic mode. Additionally, there is a demo mode for testing use; under demo mode, PC controller 210 generates signal to activate driver(s) 270 continuously without a synchronization signal. PC controller 210 is not limited to use of personal computers, as other embodiments may utilize any of a variety of computing devices with appropriate user interfaces.

Keyboard controller 220 is configured by MRI system 280 and/or US system 290, each of which may provide a synchronization signal 285 to keyboard controller 220 so that the generated waveforms are synchronized with the operation of the respective MRI or US console. Keyboard controller 220 includes signal generator 223 to provide waveform signals to actuate driver 270, which generates mechanical shear waves in object under investigation 260. The output of signal generator 223 is a waveform that is routed by switch 224 to a selected one of voltage amplifier 225 or current amplifier 226. The respective amplifier 225, 226 provides an appropriately amplified waveform to actuate driver 270. Some types of drivers are more appropriately actuated by a voltage amplifier (e.g., some piezoelectric drivers), whereas other types of drivers (e.g., some electromechanical, pneumatic, and hydraulic drivers) are more appropriately actuated by a current amplifier. Thus, system 200 includes amplifiers 225 and 226 to actuate a variety of types of drivers.

Furthermore, various embodiments are not limited to use of a single driver at a time. For instance, some embodiments are scaled to actuate two, four, six, or more drivers on an object in order to generate more complete data about the object. Drivers can be placed on different parts of the anatomy and driven at the same time, and synchronization can be performed so that the shear waves are additive at the same location. Multiple-driver embodiments may drive two or more of the same type of driver at the same time.

Another feature of keyboard controller 220 is key control panel 211, which allows a user to input system settings, such as the wave form shape, input frequency, output voltage and current, and burst count. A user may also choose a demo or triggered mode. The demo mode is described above. The triggered mode is synchronized with, and operates with, a real imaging source. Thus, at least in some respects, functionality of keyboard controller 220 overlaps somewhat with the functionality of PC controller 210. System settings and entries can be displayed to the user by display panel 222. Oscilloscope 227 displays the real time wave form of the output signal connected to the drivers. Oscilloscope 227 can be employed by a user to diagnose system problems, such as gaps in the waveform. Some embodiments are adapted to include a monitoring system and alarm that detects system problems, such as gaps in the wave form, and alerts a user of system 200.

System 200 is compatible with a variety of MRI systems (such as MRI system 280) and with a variety of US systems (such as US system 290). As shown in FIG. 2, system 200 is in communication with MRI system 280 and/or US system 290 to receive wave image data 286 therefrom. Specifically, MRI system 280 and US system 290 both produce image data that includes visualization information of the shear waves that are passed through object under investigation 260. Wave image data 286 is received by auto post-processing unit 230, which transforms wave image data 286 into an elastogram. For instance, in many embodiments, wave image data 286 is in a proprietary format of the respective MRI or US system and shows shear waves traversing the object under investigation. Auto post-processing software 230 then reformats wave image data 286 into, e.g., a standard format, such as the Data Imaging and Communications in Medicine (DICOM) format. The reformatted image is then transformed so that tissue elasticity image information is derived and depicted in a way that is understandable to a human user (e.g., with colors indicating degrees of elasticity). The elastogram is then rendered upon display 240, which includes, for example, a Liquid Crystal Display (LCD) computer monitor or other display device.

As mentioned above, system 200 is adapted for use with a variety of different imaging systems. Typically, synchronization signals are different for different manufacturers' imaging devices. Also, US and MRI systems usually use different synchronization signals, and different high- and low-field MRI devices use different synchronization signals. Thus, various embodiments of the present invention include hardware and/or software capable of interfacing with various imaging machines by conforming to a variety of synchronization signals, image data formats, and the like. Some embodiments include the interfacing functionality in software that is changeable and upgradeable independently from the hardware to maximize the ability to adapt to different, and sometimes new, imaging systems.

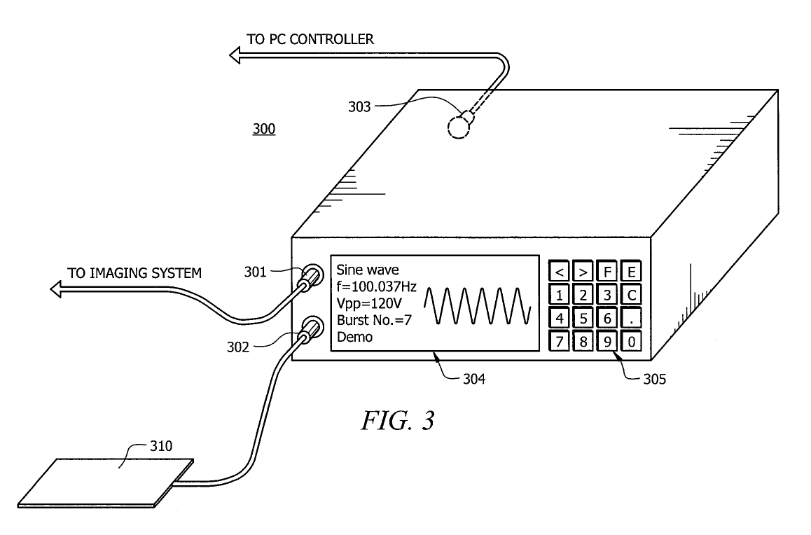

FIG. 3 is an illustration of exemplary keyboard controller 300, adapted according to one embodiment of the invention. Keyboard controller 300 shows one way to implement keyboard controller 200 of FIG. 2. Keyboard controller 300 includes input 301 adapted to receive a synchronization signal from any of a variety of MRI systems and US systems to appropriately time the waveform generator that actuates driver 310.

Keyboard controller 300 also includes output 302 for providing an amplified waveform to actuate driver 310. In some embodiments, driver 310 is included as an integral part of the elastogram system. In other embodiments, the elastogram system accommodates any of a variety of interchangeable drivers of different types. While not shown in FIG. 3, it is understood that some embodiments may include multiple output ports to accommodate multiple drivers. Input/output 303 is in communication with a PC controller (e.g., 210 of FIG. 2) so that the PC controller can display system setting information received from keyboard controller 300, and vice versa.

User input/output devices include screen 304 and keypad 305, though any type of input/output device that displays information to a user and receives input from a user can be adapted for use in various embodiments. For instance, keypad 305 can be replaced with a touch screen that is either integrated with, or separate from, display 304. Keypad 305 allows a user to enter system settings, such as waveform shape, frequency, burst count, and the like. Display 304 provides a visual indication of system settings and oscilloscope information.

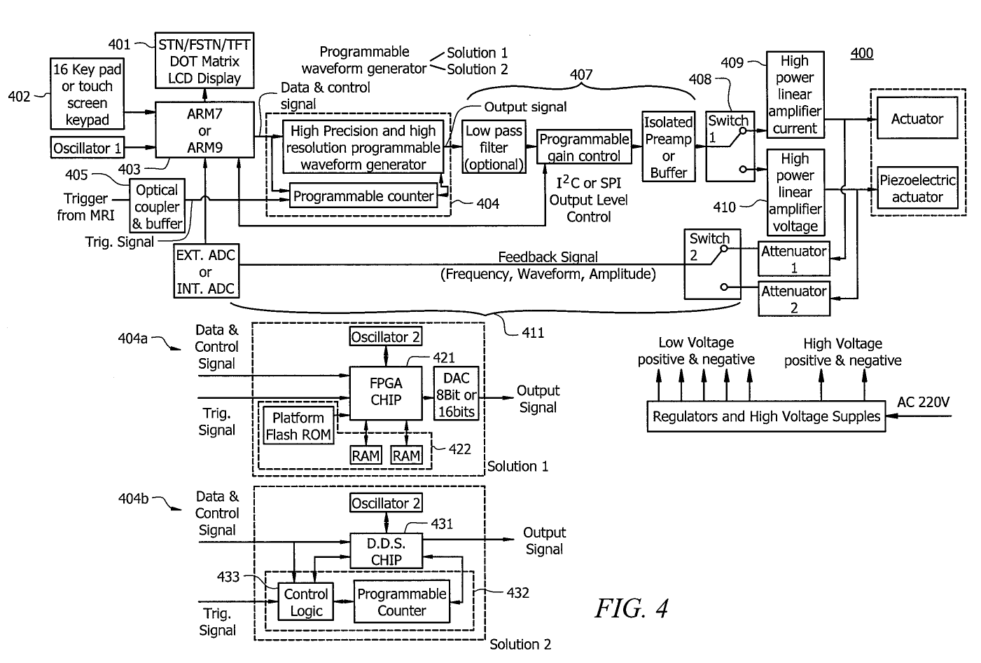

FIG. 4 is a block diagram of components of exemplary keyboard controller 400. FIG. 4 illustrates one way to implement a keyboard controller, such as keyboard controller 220 (FIG. 2), and it is understood that various substitutions, omissions, additions, and reconfigurations are possible in some embodiments.

Keyboard controller 400 includes display 401 and keypad 402, which together form a user interface assembly. The user interface assembly is controlled by processor 403, which performs other functions as well, such as implementing an oscilloscope. Waveform generator 404 performs the waveform processing, and switch and amplifier control. Waveform generator 404 receives the synchronization signal as an optical signal through optical coupler 405.

The waveform that is provided by waveform generator 404 traverses the signal path 407, where it undergoes processing that includes filtering and pre-amplification. Switch 408 is controlled by keyboard controller 400 to select one of signal amplifiers 409 and 410. As described above, some drivers are more appropriately actuated by a current amplifier, whereas other drivers are more appropriately actuated by a voltage amplifier, and keyboard controller 400, which provides selectable amplifiers, facilitates the use of any of a variety of different types of drivers. Keyboard controller 400 also includes feedback path 411, allowing processor 403 to perform control functions.

As shown in FIG. 4, there are at least two ways to implement waveform generator 404. A first option is shown as waveform generator 404a, which employs high-performance Field Programmable Gate Array (FPGA) 421. Memory 422 is used, in this embodiment, to store waveform information, such as a library of common waveforms, as well as to store other computer-readable code to provide waveform generation functionality.

A second option is shown as waveform generator 404b, which employs Direct Digital Synthesis (DDS) chip 431 and FPGA 432, which can be lower-performance FPGA than FPGA 421. FPGA 421 employs DDS chip 431 to output desired waveforms. Control logic 433 includes computer-readable code to control the waveform generation and may also include a library of saved waveforms. Various embodiments of the invention are not limited to the two options shown above. In fact, various embodiments may include any technique for waveform generation now known or later developed.

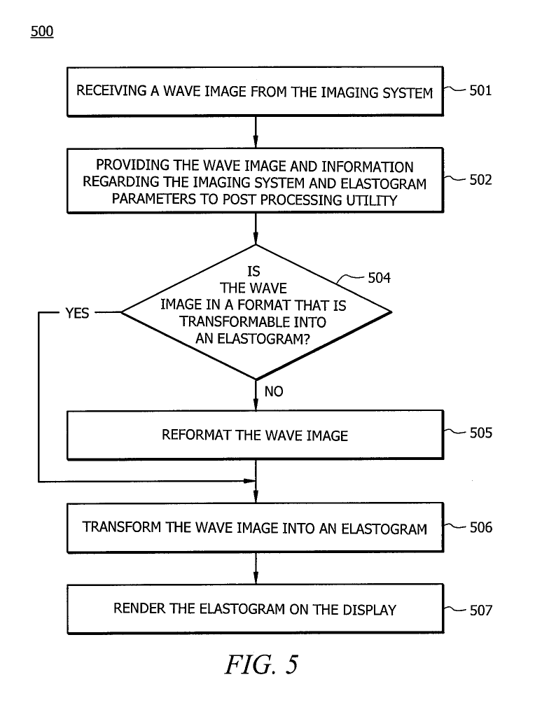

FIG. 5 is an illustration of exemplary method 500, adapted according to one embodiment of the invention, for generating an elastogram. In block 501, a wave image is received from the imaging system. For instance, the object under test is subjected to mechanical waves from a driver while the object under test is being imaged. The resulting image information from the imaging system is the wave image that is received by the elastogram system.

In block 502, the wave image and information regarding the imaging system and the elastogram parameters are provided to the post-processing utility. Elastogram parameters include the system settings (e.g., frequency of shear wave, MR or US, magnetic field of MRI), as well as tissue type and any other information helpful in transforming the wave image into an elastogram. The post-processing utility can be implemented in hardware or software by a processor executing code stored on a computer readable medium, which when executed, causes the processor to perform one or more of the actions of method 500. The post-processing utility may be implemented using a special-purpose computer or a general-purpose computer that becomes a special-purpose computer when it performs the actions of the post-processing utility.

In block 503 it is discerned whether the wave image is in a format that is transformable into an elastogram. In some instances, the wave image may be in a proprietary format that, while transformable, may not be as conveniently transformed as a wave image in a standard format. For example, a wave image in a proprietary format may be reformatted to DICOM in block 504 before it is transformed into an elastogram in block 505.

Block 505 includes transforming the wave image into an elastogram. In some examples, the wave image includes a series of images that from frame to frame show the shear waves propagating through an object under test (e.g., human tissue). The post-processing utility compares the wave image frame to frame and derives the elasticity of the object under test at a plurality of points. For instance, a shear wave traveling through tissue will typically increase its wavelength as the stiffness of the tissue increases. Hardware and/or software in the elastogram device analyzes the changes in wavelength throughout the scan and generates elasticity information therefrom. Block 505 takes into account various information when performing the transformation, such as image type (e.g., ultrasound or MRI) and properties of the image, as well as system settings. In one example, the post-processing utility receives information (either manually or automatically) about the Field of View (FOV), matrix, thickness of slice, the number of slices, and the number of phases of an MRI image. Additional information that may be used includes frequency of the shear waves, information about type and/or density of the object under test, and the like, all of which can be manually or automatically entered. Block 505 may also include various image data functionality in addition to that mentioned above, e.g., filtering.

Block 505, in some embodiments, also includes converting the image to color. For example, a red color may indicate high elasticity, a yellow color may represent lower elasticity, and a green color may represent even lower elasticity. A human user who properly perceives color can readily discern which portions of a tissue or organ show increased elasticity (often a sign of disease). In block 506, the post-processing utility renders the elastogram on the display. In some embodiments, the elastogram is rendered in real time for the user as the wave image is received. Image transformation can be performed by hardware and/or software.

Various embodiments include advantages over prior art systems. For instance, use of MRE or USE instead of X-ray imaging may reduce the amount of radiation to which a patient is exposed. Such advantage is especially pronounced with respect to the very common mammogram procedure, which entails high radiation exposure.

Additionally, increasing evidence shows the efficacy of MRE and USE in identifying some pathologies surpasses that of MRI, US, or X-ray, especially for some tumors and cirrhosis of the liver. For instance, various embodiments can provide earlier detection of cirrhosis than can the traditional fiber scan procedure. It is also envisioned that some embodiments of the invention can be used for early detection of the Alzheimer's disease, especially in the early stages of cognitive impairment, as well as lung disease and heart disease. In fact, various embodiments of the invention can be adapted to provide elastograms of any soft tissue in human and animal subjects to scan for or diagnose pathologies.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A device comprising:
a driver controller including:
a wave generator; and
an amplifier assembly receiving waves from the wave generator for driving an actuator to palpate an object under test, the amplifier assembly compatible with at least one pneumatic actuator, at least one hydraulic actuator, at least one piezoelectric actuator, and at least one electromechanical actuator;
an elastogram processor receiving a wave image of the object under test from an imaging source and generating an elastogram from the wave image; and
a user input and output assembly including:
a display rendering the elastogram; and
a user interface providing a plurality of options for selection, the options including selectable parameters to control the wave generator and to generate the elastogram.

2. The device of claim 1 wherein the amplifier assembly includes:
a current amplifier; and
a voltage amplifier.

3. The device of claim 1, wherein the selectable parameters to control the wave generator comprise one or more of the following: frequency, burst count, and output power of the amplifier assembly.

4. The device of claim 1, wherein the selectable parameters to generate the elastogram comprise one or more of the following: tissue type, actuator type, manual mode, and automatic mode.

5. The device of claim 1 wherein the elastogram process receives one or more of the parameters to control the wave generator and generates the elastogram using the received one or more of the parameters to control the wave generator.

6. The device of claim 1 integrated into a single, portable unit.

7. The device of claim 1 wherein the elastogram processor comprises:
an input compatible to receive the wave image from a Magnetic Resonance Imaging (MRI) system and ultrasound system.

8. The device of claim 1 wherein the driver controller comprises:
an oscilloscope display displaying the waves received from the wave generator.

9. The device of claim 1 wherein the driver controller comprises:
a monitor identifying defects in the wave received from the wave generator.

10. A method for generating and displaying an elastogram using an elastogram device, the elastogram device comprising a wave generator providing amplified waveforms, a user input and output assembly to control the elastogram device, and a post-processing utility to receive imaging information and create the elastogram, the elastogram device communicatively coupled to a driver associated with an object under test and communicatively coupled to an imaging device that images the object under test, the method comprising:
receiving wave image data from the imaging device, the wave image data including image data of the object under test as the object under test is subjected to mechanical waves by the driver;
automatically acquiring, by the post processing utility, one or more system settings; and
automatically generating the elastogram based at least in part on the wave image data and the acquired system settings.

11. The method of claim 10 further comprising:
reformatting the wave image data into a standard format in response to discerning a proprietary format of the wave image data.

12. The method of claim 10 further comprising:
discerning a type of the driver; and
selecting a first one of a plurality of amplifiers corresponding to the driver type.

13. The method of claim 12 further comprising:
replacing the driver with another driver;
discerning a type of the other driver;
selecting a second one of the plurality of amplifiers corresponding to the driver type of the other driver.

14. The method of claim 10 further comprising:
discerning a type of the imaging device, and wherein generating the elastogram comprises:
generating the elastogram based at least on part on the discerned type of the imaging device.

15. The method of claim 10 wherein the imaging device is selected from the list consisting of:
an ultrasound device; and
a magnetic resonance imaging device.

16. The method of claim 10 wherein the acquired system settings include one or more of wave burst count, wave frequency, output voltage of the wave generator, output current of the wave generator, and waveform type.

17. The method of claim 10 wherein the post-processing utility further acquires tissue characteristic information and uses the tissue characteristic information to generate the elastogram.

18. The method of claim 10 wherein generating the elastogram comprises:
transforming the wave image data to derive and display elasticity information; and
indicating degrees of elasticity through color variation.

19. An integrated elastogram device comprising:
a driver controller including:
a wave generator; and
an amplifier assembly receiving waves from the wave generator for driving an actuator to provide mechanical waves to an object under test;
a user interface providing a plurality of options for selection, the options including selectable parameters to control the wave generator and to generate an elastogram
an imaging engine including:
an elastogram processor automatically receiving at least one of the selectable parameters and automatically generating the elastogram from wave image data of the object under test received from an imaging source and the received selectable parameters; and
a display rendering the elastogram.

20. The integrated elastogram device of claim 19 wherein the selectable parameters include one or more of wave burst count, wave frequency, output voltage of the wave generator, output current of the wave generator, and waveform type.

21. The integrated elastogram device of claim 19 wherein the elastogram processor includes a computer processor executing a program to transform the wave image data into the elastogram.

22. The integrated elastogram device of claim 19 wherein the elastogram represents elasticity for a plurality of locations in a human tissue.

23. An elastogram system comprising:
an elastogram processing engine compatible with a Magnetic Resonance Imaging (MRI) system and an ultrasound imaging system receiving a wave image from an imaging source, the elastogram engine transforming the wave image received from the imaging source to generate an elastogram; and
a display communicatively coupled to the elastogram processing engine and displaying the elastogram.

24. The elastogram system of claim 23 further comprising:
a driver controller including:
means for generating waveforms; and
means for receiving waveforms from the waveform generating means, amplifying the received waveforms, and driving an actuator with the amplified received waveforms.

25. The elastogram system of claim 24 wherein the actuator palpates a tissue under test in the imaging source.

26. The elastogram device of claim 24 integrated into a single, portable unit.
